(19) United States
(12) Patent Application Publication
(10) Pub. No.: US 2005/0288548 A1
(43) Pub. Date: Dec. 29, 2005

(54) Endoscope with limited rotation shaft joint

(75) Inventor: Dennis R. Boulais, Danielson, CT (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: SCIMED Life Systems, Inc.

(21) Appl. No.: 10/879,370

(22) Filed: Jun. 29, 2004

(51) Int. Cl. A61B 1/00

(52) U.S. Cl. 600/102; 600/137

(57) ABSTRACT

An endoscope with a limited rotation shaft joint that limits the amount of shaft rotation and the chance of twisting the endoscope cables, tubes, and controls to where they break or perform improperly. Shaft joint components can be designed to allow a 0°-360° degree of rotation. The limited rotation shaft joint includes a shaft adapter with an annular slot and a pin or molded stop detail. A housing is provided with a hole for the shaft adapter and a thumb screw. Once assembled, the shaft adapter is placed within the hole in the housing, and the thumb screw is twisted so that its end extends into the annular slot to hold the shaft adapter in place, causing the pin or molded stop detail to eventually hit the thumb screw to halt rotation. The placement, width, or number of pins or stop details determines the amount of allowable shaft rotation.

### FIELD OF THE INVENTION

The present invention relates to medical devices, and in particular to endoscopes.

### BACKGROUND OF THE INVENTION

Endoscopic procedures for treating abnormal pathologies within the alimentary canal system and biliary (including the biliary, hepatic, and pancreatic ducts) are increasing in number. The endoscope provides access to the general area of a desired duct using direct visualization. In general, for treatment of an abnormal pathology within a patient's biliary tree, an endoscope is first introduced into the mouth of the patient. The endoscope includes a proximal end and a distal end, and has a lumen extending longitudinally between the proximal and distal ends. The endoscope is guided through the patient's alimentary tract or canal until an opening at the distal end of the endoscope is proximate to the area to receive treatment. At this point, the endoscope allows other components, such as a catheter, to access the targeted area.

In connection with the endoscope, an operator control module is typically provided that allows a user to control and steer the operation of the endoscope. In general, the endoscope shaft must be rotated during the course of endoscopic procedures in order to align the working portions of the endoscope tip to perform functions (e.g., polyp removal, drainage, etc.). In general, the cables that manipulate the endoscope tip are terminated inside the operator control module and thus limit the amount of rotation that can be performed without damaging the cables, tubes, and controls. Thus, one drawback for endoscopes with unlimited available rotation is that they may be rotated so far so as to twist the cables, tubes, and controls so far that they break or no longer perform properly.

The present invention is directed to an apparatus that overcomes the foregoing and other disadvantages. More specifically, the present invention is directed to an apparatus that limits the rotation of the endoscope shaft so as to prevent damage to the cables, tubes, and controls of the endoscope.

### SUMMARY OF THE INVENTION

The present invention is directed to an endoscope with a limited rotation shaft joint. In accordance with one aspect of the invention, the limited rotation shaft end joint provides a way to limit the amount of shaft rotation and the chance of twisting the cables, tubes, and controls so far that they break or no longer perform properly. The shaft is allowed to rotate inside the mounting joint at the control console or at the breakout box. The advantage of using a limited rotation shaft end joint, is that unlike a design where a low torque shaft is allowed to twist about its central axis with both ends of the shaft being fixed to the control console or the breakout box, the limited rotation shaft end joint provides a way to limit the amount of shaft rotation.

In accordance with another aspect of the invention, the parts of the shaft joint are designed to allow any degree of rotation. In one embodiment, the available degrees of rotation may range from 0° to up to 360°.

In accordance with another aspect of the invention, a shaft adapter is provided with an annular slot. A pin or molded stop detail is provided within the annular slot. A thumb screw that is provided through a housing around the shaft and shaft adapter slides into the annular slot and holds the shaft adapter in place. As the shaft rotates, the pin or molded stop detail hits the thumb screw and thus limits the rotation. Thus, the placement of the stop detail and the width or number of the details determines the total amount of rotation that is allowed.

In accordance with another aspect of the invention, a break or locking mechanism can be provided that allows the operator to lock the shaft in the joint and prevent any rotation of the shaft in the mounting. This may be a preferred embodiment for certain applications.

In accordance with another aspect of the invention, in one embodiment, a preferred configuration is to allow 180° of shaft rotation. This provides enough rotation to spin the working channel port into position from any starting orientation with respect to a polyp.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a diagram illustrating a shaft adapter with an annular slot and a molded stop detail in accordance with the present invention;

FIG. 2 is a diagram illustrating a housing with a thumb screw and a hole for the shaft adapter of FIG. 1; and

FIG. 3 is a diagram illustrating the assembly of the shaft adapter of FIG. 1 into the hole of the housing of FIG. 2.

### DETAILED DESCRIPTION OF THE PREFERRED EMBODIMENT

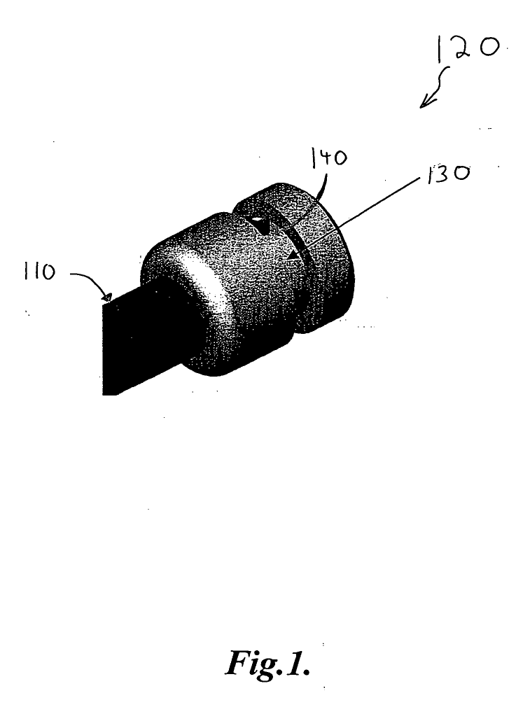

FIG. 1 is a diagram of a shaft adapter 120 that is formed in accordance with the present invention. The shaft adapter 120 is placed over a shaft 110. The shaft adapter 120 includes an annular slot 130 and a pin or molded stop detail 140. As will be described in more detail below, the pin or molded stop detail 140 is utilized to limit the rotation of the shaft 110.

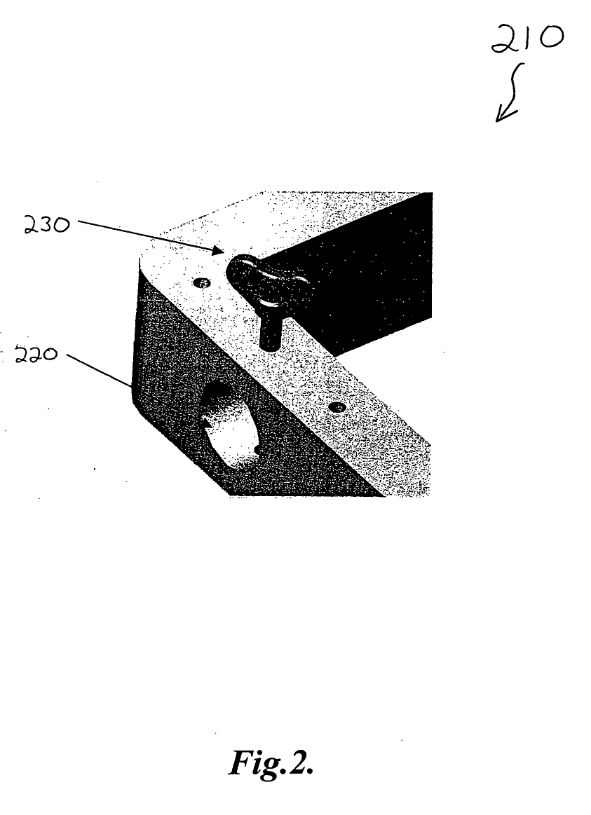

FIG. 2 is a diagram of a housing 210. The housing 210 includes a hole 220 which is intended to hold the shaft adapter 120 of FIG. 1. The housing 210 also includes a thumb screw 230. When assembled, the end of the thumb screw 230 is intended to extend into the annular slot 130 of the shaft adapter 120.

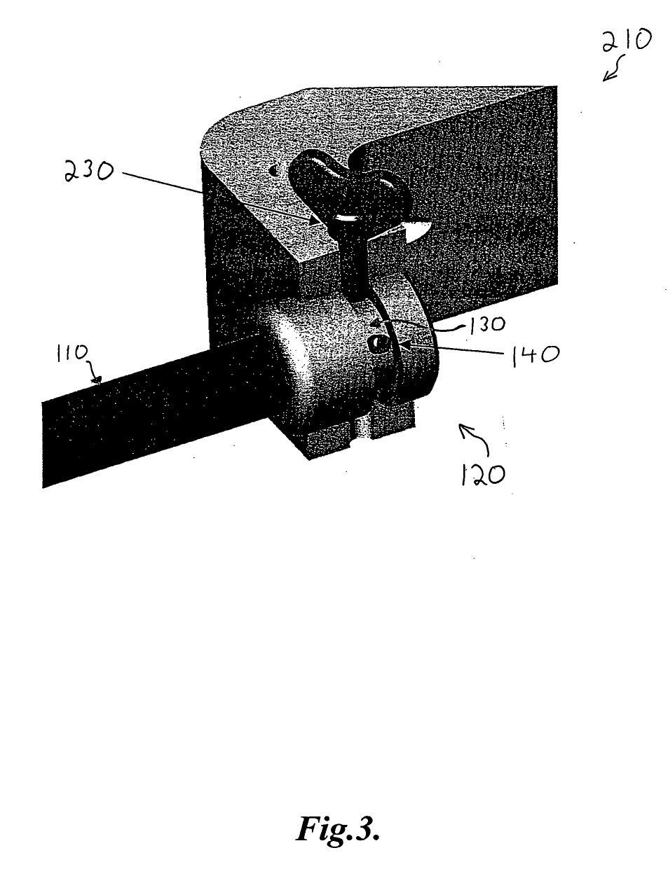

FIG. 3 is a diagram illustrating the assembly of the shaft adapter 120 into the hole 220 of the housing 210. As illustrated in FIG. 3, the thumb screw 230 has been twisted into the housing 210 until its end extends into the annular slot 130. By extending into the annular slot 130, the thumb screw effectively holds the shaft adapter 120 in place. As the shaft 110 rotates, the pin or molded stop detail 140 hits the end of the thumb screw 230 and thus stops the shaft 110 from rotating. The placement of the pin or molded stop detail 140 and the width or number of the pins or molded stop details (in other embodiments there may be more than one) thus determines the total amount of rotation of the shaft 110 that is allowed. The parts of the shaft joint can be designed to allow any degree of rotation, from 0° up to 360°. In one embodiment, 180° of shaft rotation is allowed. This provides enough rotation to spin the working channel port into position from any starting orientation with respect to a polyp.

In one embodiment, a brake or locking mechanism may also be provided. The brake or locking mechanism allows the operator to lock the shaft in the joint and prevent any rotation of the shaft in the mounting. This may be a preferred means of operation for some users.

The above-described configuration by which the shaft is allowed to rotate inside the mounting joint at the control console or at the breakout box is in contrast to certain other known endoscope configurations, where the axial rotation of the endoscope shaft with respect to the control console or the breakout box is accomplished by letting the shaft twist about itself. In other words, in certain alternative designs, a low torque design shaft is allowed to twist about its central axis with both ends of the shaft being fixed to the control console or the breakout box. The advantage of the present invention which uses a limited rotation shaft end joint is that it provides a way to limit the amount of shaft rotation and thus reduces the chance of twisting the cables, tubes, or controls of the endoscope so far that they break or no longer perform properly.

While the preferred embodiment of the invention has been illustrated and described, it will be appreciated that various changes can be made therein without departing from the spirit and scope of the invention.

1. A rotation limiting system which limits the rotation of an endoscope shaft, the rotation limiting system comprising:
a rotation limiting device for limiting rotation beyond a specified position;
a housing with a hole for receiving the rotation limiting device, the rotation limiting device being allowed to rotate in a first direction within the hole until the specified position is reached, and once the specified position is reached the rotation limiting device limiting further rotation of the endoscope shaft in the first direction.

2. The system of claim 1, wherein the rotation limiting device comprises a shaft adapter.

3. The system of claim 2, wherein the shaft adapter comprises an annular slot.

4. The system of claim 3, wherein the shaft adapter further comprises a stop element which is located within the annular slot.

5. The system of claim 4, wherein the stop element comprises a pin.

6. The system of claim 4, wherein the stop element is molded.

7. The system of claim 1, wherein the housing includes an extension element which extends into the hole.

8. The system of claim 7, wherein the extension element is a thumb screw.

9. The system of claim 7, wherein when the rotation limiting device is assembled into the hole in the housing, the extension element extends into an annular slot in the rotation limiting device.

10. The system of claim 9, wherein the rotation limiting device further comprises a stop element that is located within the annular slot, the stop element stopping the rotation of the rotation limiting device when it is rotated so as to come in contact with the extension element.

11. In an endoscope system, an improvement for limiting the rotation of an endoscope, the endoscope being rotatable so as to allow an operator to orient elements at the distal end of the endoscope in desired positions, the improvement comprising:
a rotation limiting device, the rotation limiting device comprising a stop element for limiting the rotation of the endoscope shaft.

12. The improvement of claim 11, wherein the rotation limiting device is adjustable so as to adjust the amount of rotation that is allowed for the endoscope shaft.

13. The improvement of claim 11, wherein the rotation limiting device comprises a shaft adapter.

14. The improvement of claim 13, wherein the shaft adapter comprises an annular slot.

15. The improvement of claim 14, wherein the stop element is located within the annular slot.

16. The improvement of claim 15, further comprising a housing with a hole with an extension element, the hole receiving the rotation limiting device, the extension element extending into the annular slot and thus helping to maintain the rotation limiting device in position within the hole, and the extension element limiting further rotation of the rotation limiting device when it is rotated such that the stop element comes in contact with the extension element.

17. A rotation limiting device for an endoscope, comprising:
a circular end portion that is rotatable within a hole that has an extension element; and
a stop element that extends outward from at least a portion of the circular end portion, the stop element limiting further rotation of the circular end portion when it comes into contact with the extension element.

18. The device of claim 17, wherein the circular end portion comprises a slot in which the stop element is located and into which the extension element extends.

19. The device of claim 17, wherein the stop element comprises a plurality of stop elements.

20. The device of claim 17, further comprising a locking mechanism which allows an operator to lock the shaft so as to prevent any further rotation of the shaft.
